(19) United States
(12) Patent Application Publication
(10) Pub. No.: US 2001/0053777 A1
(43) Pub. Date: Dec. 20, 2001

(54) Drug treatment for restless leg syndrome

(76) Inventor: Hans Michael Brecht, Ingelheim (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/919,731

(22) Filed: Aug. 1, 2001

Related U.S. Application Data
(62) Division of application No. 09/639,291, filed on Aug. 15, 2000, now abandoned.

(30) Foreign Application Priority Data
Aug. 19, 1999 (DE) DE 199 38 823.7

(51) Int. Cl. A61K 31/5513; A61K 31/4985; A61K 31/48; A61K 31/485; A61K 31/4745

(52) U.S. Cl. 514/221; 514/282; 514/249; 514/293; 514/288

(57) ABSTRACT

A method for the treatment of Restless Leg Syndrome (RLS), which comprises administering an α2-agonist and a second agent selected from the group consisting of the dopamine agonists, opioids, benzodiazepines and the combination of L-DOPA plus a decarboxylase inhibitor.

### RELATED APPLICATIONS

This application is a division of Ser. No. 09/639,291 filed Aug. 15, 2000.

### FIELD OF THE INVENTION

The invention relates to a new combination of active substances for more effective treatment of Restless Leg Syndrome (RLS) consisting of an α2-agonist and another neuropsychic drug which reduces the symptoms of RLS as a monotherapy.

### BACKGROUND TO THE INVENTION

Restless Leg Syndrome is a neurological disorder which manifests itself chiefly as sensory disorders of the legs such as tingling, dragging, tearing, itching, burning, cramp or pain and in those affected triggers an irresistible compulsion to move. Frequently these disorders occur when the affected person is resting. Particularly at night, during sleep, these sensory disorders and the consequent compulsive movements lead to restlessness and sleep disorders.

RLS occurs at all ages, increasing in frequency at more advanced ages. The prevalence in the general population is about 5%. Because of the characteristics of the symptoms RLS is one of the most common causes of sleep problems. RLS is the cause of sleeping and waking problems in 7% of 20-40 year-olds, 18% of 40-60 year-olds and 33% of over 60s.

When the patient's quality of sleep or life is increasingly affected by RLS or the patients suffer from daytime tiredness, treatment is indicated. The need for treatment generally sets in at the age of 40-50.

Hitherto there has been no permitted drug treatment available. In therapy trials, monotherapies with dopamine agonists, opiates, benzodiazepines, carbamazepine, clonidine or the combined administration of laevodopa (L-DOPA) in conjunction with a dopadecarboxylase inhibitor have had mixed degrees of success. Most studies have been done on the use of L-DOPA in RLS. In long-term therapy there is a significant alleviation of the complaint, with an improvement in the quality of life and sleep. The disadvantage of the L-DOPA therapy, however, is that in many patients the effectiveness declines and/or there is a shift of the RLS problems to the morning (rebound) or afternoon (augmentation).

For individual dopamine agonists short-term therapy trials have been conducted. The dopamine agonists investigated include: bromocryptine, cabergoline, alpha-dihydroergocryptine, lisuride, pergolide, pramipexole and ropinirol. All these dopamine agonists were found to be effective. The results of trials on long-term therapy with dopamine agonists are not yet available, so the question of the loss of activity after long-term use (tachyphylaxis) cannot be answered yet.

The disadvantage of the dopamine agonists is the incidence of side-effects such as nausea, vomiting, dizziness, hypotension, constipation and sleeplessness, which generally occur initially and in dose-dependent manner.

The use of the anti-Parkinson's drug pramipexole, (S)-2-amino-6-n-propylamino-4,5,6,7-tetrahydro-benzothiazole, a D2/D3 agonist, for treating RLS is described in WO 98/31362, to which reference is hereby made in its entirety.

Benzodiazepines and opiates are also effective in RLS. Because of the risk of dependency and the build-up of tolerance, however, these substances are only available for therapy on a restricted basis.

Carbamazepine has only been tested on RLS in a few partly open trials. It gives only partial relief from the complaint and is not currently viewed as a suitable drug for treating RLS.

The effect of clonidine, 2-(2,6-dichloroanilino)-4,5-dihydroimidazole, which was originally developed as an antihypertensive and miotic, in the treatment of RLS has been studied in 4 open trials, 2 double-blind, placebo-controlled trials and a single case study. The daily doses were between 0.1-0.9 mg. The patients reported a (statistically significant) reduction in perceived symptoms such as paresthesia, compulsive movement and tiredness during the day. According to the objective polysomnographic measuring parameters, the sleep latency was indeed shortened, but the quality of sleep, frequency of waking or periodic leg movements in sleep (PLMS) were not affected. Since substances are available which are more effective as monotherapies, clonidine is currently only recommended as an alternative form of therapy under certain circumstances.

A further disadvantage of most monotherapies is that the quantity of the active substance in question has to be increased over time in order to ensure therapeutic success.

### SUMMARY OF THE INVENTION

Surprisingly, it has now been found that the combined administration of an α2-agonist together with another neuropsychic drug which also leads to a reduction of the RLS symptoms in monotherapy, unexpectedly suppresses the RLS symptoms synergistically. In fact, it has been found that in combination each of the two active substances can be used in a significantly lower dose that when they are used in monotherapy. In combination therapy a more significant improvement in the condition of the RLS patient is achieved within a short time than was achieved by the relevant monotherapy, even if the latter was carried out over a lengthy period and with fairly high doses.

### DETAILED DESCRIPTION OF THE INVENTION

The present invention provides, as its first aspect, a novel method for the treatment of Restless Leg Syndrome which comprises administering both an α2-agonist and another neuropsychic drug which also leads to a reduction in RLS symptoms in monotherapy. As a second aspect, the invention provides a novel pharmaceutical composition suitable for the treatment of Restless Leg Syndrome which comprises both an α2-agonist and another neuropsychic drug which also leads to a reduction in RLS symptoms in monotherapy.

The combination according to the invention overcomes the disadvantages of the monotherapies known from the prior art.

Another advantage of the invention is that in this combination the α2-agonist synergistically influences the effect of the other neuropsychic drug known from the RLS-monotherapy (or vice versa) by increasing the activity, so that even low doses of the two active substances are enough to improve the patient's comfort without any intolerable side-effects occurring. In addition, the combined administration of these two active substances leads to better responses and a higher response rate in patients with RLS.

Imidazole receptor agonists are preferred as the α2-agonist. Also preferred are azepexol, brimonidine, clonidine, dexmedetomidine, lofexidine, medetomidine, moxomidine, rilmenidine, talipexol, tiamenidine, tizanidine, AGN-190837, AGN-193080, BAM 1110, BAM-1125, CHF-1035, MPV-295, MPV-2426, S-18616, UK-1403.

Of these, the following are preferred: brimonidine, clonidine, dexmedetomidine, lofexidine, moxomidine, talipexol, AGN-193080, BAM-1125, MPV-2426. Clonidine is particularly preferred. In each case, a pharmacologically acceptable salt or an ester or a prodrug form, e.g. an ester, may also be used as the active substance. The same applies to all the active substances listed in the context of this invention.

The additional neuropsychic drug is preferably an opioid, a benzodiazepine, a dopamine agonist or a combination of laevodopa (L-DOPA) plus decarboxylase inhibitor.

From the group comprising levodopa (L-DOPA) plus decarboxylase inhibitor, the combinations of L-DOPA plus benserazide and L-DOPA plus carbidopa are particularly preferred.

Of the dopamine agonists, bromocryptine, cabergoline, α-dihydroergocryptine, lisuride, pergolide, piripedil, pramipexole (HCl), ropinirol, S(−)-2-(N-propyl-N-2-thienylethylamino)-5-hydroxy-tetraline (e.g. as N-0923) or (R)-5,6-dihydro-5-(methylamino)-4H-imidazo(4,5-ij)-quinolin-2(1H)-one R-6 (PNU 95666) or a pharmacologically acceptable salt thereof are preferred. Particularly preferred are: cabergoline, pergolide, piripedil, pramipexole, pramipexole hydrochloride, ropinirol, N-0923 and PNU 95666. Most preferred are pramipexole and pramipexole hydrochloride.

Of the opioids, buprenorphine, codeine, dextropropoxyphen, dihydrocodeine, fentanyl, hydromorphone, laevomethadone, morphine, oxycodon, pethidine, propoxyphen, sufentanyl, tilidine, tilidine/naloxone, tramadol or the pharmacologically acceptable salts thereof are preferred. Particularly preferred are dihydrocodeine, oxycodon, tilidine/naloxone, tramadol, propoxyphen and morphine.

Of the benzodiazepines, clonazepam, temazepam, nitrazepam, oxazepam and brotizolam are preferred. Clonazepam is particularly preferred.

The following combinations are preferred:

1. clonidine, or a pharmacologically acceptable salt thereof with L-DOPA plus decarboxylase inhibitor

2. clonidine with a dopamine agonist, the combination with pramipexole or a pharmacologically acceptable salt thereof being particularly preferred.

3. clonidine with a benzodiazepine, more preferably with clonazepam.

4. clonidine with an opioid, more preferably with tilidine/naloxone

5. In severe cases a triple combination of clonidine plus L-DOPA or dopamine agonist plus opioid or benzodiazepine is advisable.

The combination of active substances according to the invention may be formulated according to the current pharmaceutical methods known from the prior art so that they can be administered by oral, spinal, anal or intravenous route or by inhalation, subcutaneously or transdermally. Oral and transdermal preparations are preferred.

The preparation may be given orally in the form of a tablet, powder, powder in a capsule (e.g. a hard gelatine capsule), as a solution or suspension. For spinal, intravenous and subcutaneous applications, the combination of active substances according to the invention is given as a solution. The preparation may be administered anally in suppositories. For inhalation, the combination of active substances may be given as a powder, as an aqueous or aqueous-ethanolic solution or using a propellant gas formulation. For transdermal administration the active substance may be applied to the skin as an ointment or cream, but is preferably applied by means of a plaster.

In the case of plasters, the active substance or combination of active substances is either released directly onto the outer layer of the skin or is released directly into the underlying layers of the skin using a transdermal plaster, in the form of a solution or a gel, e.g. embedded in a polymer matrix, through micro-pins or micro-cutters which penetrate the horny layer of the skin. A transdermal plaster with micro-pins or micro-cutters of this kind is disclosed for example in patent application WO 97/03718. Patent application WO 91/07998 describes a process by means of which active substances can be applied more satisfactorily transdermally by adjusting the skin to a specific pH. U.S. Pat. No. 5,112,842, or the corresponding European Patent EP 0428038, discloses a transdermal plaster for administering pramipexole. Reference is hereby made expressly to the contents of all three patents, to show how the combination of active substances according to the invention can be applied using a transdermal plaster.

Both types of plaster described above (with and without microcutters or micropins) release the active substance continuously onto or into the skin, so as to avoid concentration peaks and the possible side effects associated with them. The active substance or combination of active substances can be released passively or actively. Active transfer can be by purely mechanical means, electrically, osmotically or by iontophoresis. If desired, the release may be controlled electronically, optionally with monitoring of the blood plasma level by sensors or microsensors which are integrated in the plaster or communicate therewith, as a result of which the blood plasma level can be adjusted deliberately to suit individual requirements and consequently a steady release is not absolutely essential.

In every case, the two active substances may be formulated separately (e.g. in a capsule or as a tablet), in a single formulation but separate from one another (e.g. in a capsule with two or more chambers) or mixed together in a single formulation (e.g. in the form of a tablet or in a capsule with only one chamber).

When the two active substances are formulated separately independently of one another, it is not essential for the two substances to be administered by the same route of administration; rather, combinations of formulations may be used wherein the two active substances are administered by separate routes. For the combination of clonidine and pramipexole, for example, clonidine may be given orally while pramipexole is administered transdermally, e.g. using the transdermal plaster described above. However, those formulations wherein the two active substances are administered by the same route are preferred. The two active substances are advantageously administered together in one preparation.

In the case of the transdermal plasters, the two active substances may be administered, for example, either in separate plasters, in a joint plaster in which the two active substances are stored separately within the plaster, or they may be mixed together in one plaster. The same is also true of the other administration forms described above.

The active substance formulation according to the invention is prepared by the methods known from the prior art, depending on the method of administration, and may accordingly contain the formulation constituents known in the art. They may also contain other pharmacologically active substances or cosmetic additives.

In every case, the two active substances from the group of the ∀2-agonists and the other neuropsychic drugs may be used both as neutral compounds or in the form of a pharmacologically acceptable salt. The two active substances may be used both as neutral compounds and as two identical or two different salts or as a combination of a salt of one active substance and the other, neutral, active substance. The different variants are influenced by the method of administration. Where the two active substances are provided in a joint formulation, this is preferably the neutral compound or the same salt (e.g. the hydrochloride). The same is also preferably true when the two active substances are taken orally as tablets or capsules.

Independently of the method of administration, the active substances are preferably administered simultaneously or within an overlapping time frame. In the case of oral administration they should be taken within 1 hour, preferably within 15 minutes of each other.

The amount of individual active substance per single dose, in relation to the neutral compound, (unless otherwise stated) corresponds to an oral administration of:

α2-agonist

The dosage of the α2-agonist corresponds to an oral administration of 0.001-15 mg, preferably 0.001-10 mg, more preferably 0.01 to 5.0 mg and most preferably 0.01 mg-1 mg.

For the following active substances the following doses are preferred, the amount in each case corresponding to an orally administered single dose of the neutral compound: azepexol: 0.5 to 10.0 mg, preferably 3.0 to 7.0 mg, more preferably 4.5 to 5.5 mg, clonidine: 0.01 to 1.0 mg, preferably from more than 0.01 to 0.5 mg and most preferably from 0.05 to 0.3 mg, lofexidine: 0.05 to 5.0 mg, preferably 0.05 to 3.0 mg, more preferably 0.1 to 2.0 mg, rilmenidine: 0.05 to 5.0 mg, preferably 0.05 to 3.0 mg, more preferably 0.1 to 2.0 mg, tiamenidine: 0.05 to 7.0 mg, preferably 0.1 to 5.0 mg, more preferably 0.5 to 3.5 mg.

Levodopa Plus Decarboxylase Inhibitor:

L-DOPA in combination with benserazide: 10 to 500 mg, preferably 50-200 mg and particularly preferably from 100-200 mg of L-DOPA and 1-100 mg, preferably 10-50 mg and particularly preferably 25-50 mg of benserazide, L-DOPA in combination with carbidopa: 10 to 500 mg, preferably 10-300 mg and particularly preferably from 50-200 mg of L-DOPA and 1-100 mg, preferably 10-50 mg and particularly preferably 12.5-50 mg of carbidopa;

Dopamine Agonists

Bromocryptine: 1.25-20.0 mg, preferably 2.5-15.0 mg,

Cabergoline: 0.05-5.0 mg, preferably 0.5-3.0 mg,

α-Dihydroergocryptine: 5-60 mg, preferably 10-40 mg,

Lisuride: 0.1-5 mg, preferably 0.1-1.0 mg,

Pergolide: 0.05-1.0 mg, preferably 0.1-1.0 mg,

Pramipexole (HCl): 0.01-5.0 mg, preferably 0.1-1.5 mg, more preferably 0.125-1.0 mg

Ropinirol: 0.2-10.0 mg, preferably 0.25-6.0 mg;

Opioids

Codeine: 10 to 100 mg, preferably 15-60 mg,

Dihydrocodeine: 10 to 100 mg, preferably 40-80 mg,

Oxycodon: 4.5-20 mg

Propoxyphen: 65-300 mg

Tilidine/Naloxone: 40-60/3-5 mg

Tramadol: 10 to 500 mg, preferably 25 to 200 mg and more preferably from 50-100 mg,

Morphine: 1 to 500 mg, preferably 1 to 200 mg and more preferably from 10-100 mg,

Benzodiazepines

Clonazepam: 0.01-10 mg, preferably, 0.1-5 mg and more preferably from 0.25-2.0 mg,

Brotizolam: 0.01-2 mg, preferably, 0.05-0.5 mg and more preferably from 0.1-0.3 mg.

Temazepam: 15-30 mg

Nitrazepam: 5-10 mg

Oxazepam: 10-20 mg

For transdermal use, because of the continuous method of administration, a different quantity may be given to achieve a correspondingly effective blood plasma concentration. The exact amount of active substances can be determined by simple tests, depending on the method of administration.

### EXAMPLE

2 patients with RLS (55 year old man and 67 year old woman) were treated with a combination therapy of pramipexole and clonidine.

1. Therapeutic History:

Both patients had been suffering from severe sleep disorders for more than 15 years and had previously been treated with L-DOPA, benzodiazepines (brotizolam, oxazepam), carbamazepine and bromocryptine or pergolide. The symptoms (discomfort, cramps and pains in the legs, compulsive movement, problems falling asleep and sleeping through, as well as daytime tiredness and feelings of exhaustion) improved significantly, but the two patients were never free from symptoms. In both patients, L-DOPA led to typical augmentation during the day which disappeared when they switched to a dopamine agonist. It was not possible to increase the dose of pergolide or bromocryptine any further because of side effects such as nausea, gastrointestinal problems and dizziness. Brotizolam and oxazepam improved the falling asleep and sleeping through, in particular, but these two substances could only be prescribed for a limited time on account of the risk of dependency.

After the previous therapy had been brought slowly and completely to an end the two patients were treated with pramipexole in an amount of 0.088 mg two hours before bedtime. In the male patient, the daily dose had to be increased to 0.36mg at weekly intervals, whilst in the female patient it had to be increased to 0.27mg. The symptoms certainly improved in both patients, but the two patients did not report any difference from their earlier therapy.

The pramipexole was slowly reduced in both patients and finally stopped and a therapy trial with clonidine was started. The clonidine was also initially prescribed in a single dose of 0.075 mg two hours before bedtime and increased by 0.075 mg at intervals of 3 days.

The male patient was finally given 0.225 mg, the female patient 0.45 mg of clonidine hydrochloride as a single dose before bedtime; both patients stated that they felt hardly any paresthesia and the compulsive movements had also improved, but the quality of sleep and the number of times they woke during the night had not changed. As a result of some intolerable side effects such as dry mouth, dizziness and constipation, both patients asked if they could stop taking the clonidine.

2. Treatment with a combination therapy of clonidine and pramipexole After slowly bringing the clonidine therapy to a complete halt and after a treatment-free period of about 1 week, both patients were treated with a combination of 0.088 mg of pramipexole and 0.075 mg of clonidine. From the very first night, both patients reported a significant alleviation of their symptoms. After 7 days the dosage of pramipexole had been increased to 0.18 mg and the dosage of clonidine to 0.15 mg, two hours before going to nd sleep. At the end of the 2 week of treatment, both patients reported that virtually all their subjective symptoms such as tingling, cramp, pain in the legs, restlessness of the legs during the night, problems on going to sleep and sleeping through were no longer present or had been reduced to a tolerable minimum, so that their daily quality of life was no longer impaired. The combined administration of pramipexole and clonidine showed no reduction in activity in either patient right to the end of the observation period of about 3 months.

What is claimed is:

1. A method for treating restless leg syndrome which comprises administering a therapeutic amount of an α2-agonist and a second agent selected from the group consisting of the dopamine agonists, opioids, benzodiazepines and the combination of L-DOPA plus a decarboxylase inhibitor.

2. The method according to claim 1, wherein the quantity of the α2-agonist per single dose based on the neutral compound corresponds to an oral dose of 0.001-15 mg.

3. The method according to claim 1 wherein the α2-agonist is an imidazole agonist.

4. The method according to claim 1, wherein the α2-agonist is selected from the group consisting of azepexol, brimonidine, clonidine, dexmedetomidine, lofexidine, medetomidine, moxomidine, rilmenidine, talipexol, tiamenidine, tizanidine, AGN-190837, AGN-193080, BAM 1110, BAM-1125, CHF-1035, MPV-295, MPV-2426, S-18616, and UK-1403, and the pharmacologically acceptable salts thereof.

5. The method according to claim 4, wherein the quantity based on the neutral compound per single dose corresponds to
an oral dose of 0.01 to 1.0 mg, for clonidine;
an oral dose of 0.5 to 10.0 mg, for azepexol;
an oral dose of 0.05 to 5.0 mg, for lofexidine;
an oral dose of 0.05 to 5.0 mg, for rilmenidine; and
an oral dose of 0.05 to 7.0 mg, for tiamenidine.

6. The method according to claim 1 wherein, wherein the α2-agonist is selected from the group consisting of azepexol, brimonidine, clonidine, dexmedetomidine, lofexidine, medetomidine, moxomidine, rilmenidine, talipexol, tiamenidine, tizanidine, AGN-190837, AGN-193080, BAM 1110, BAM-1125, CHF-1035, MPV-295, MPV-2426, S-18616, and UK-1403, or a pharmacologically acceptable salt thereof and the second agent is selected from the group consisting of the dopamine agonists, opioids, benzodiazepines and the combination of L-DOPA plus a decarboxylase inhibitor.

7. The method according to claim 1, wherein the a2-agonist is clonidine or a pharmacologically acceptable salt thereof.

8. The method according to claim 1, wherein the second agent is a combination of L-DOPA or a pharmacologically acceptable salt thereof and a decarboxylase inhibitor.

9. The method according to claim 8, wherein the decarboxylase inhibitor is benserazide or carbidopa or a pharmacologically acceptable salt thereof.

10. A pharmaceutical composition according to claim 9, wherein the quantity of L-DOPA in combination with benserazide corresponds to an oral dose of 10 to 500 mg of L-DOPA, and 1-100 mg of benserazide, and for L-DOPA in combination with carbidopa it corresponds to an oral dose of 10 to 500 mg of L-DOPA and 1-100 mg of carbidopa.

11. The method according to claim 1, wherein the second agent is a dopamine agonist.

12. The method according to claim 11, wherein the dopamine agonist is selected from the group consisting of bromocryptine, cabergoline, α-dihydroergocryptine, lisuride, pergolide, piripedil, pramipexole (HCl), ropinirol, S(−)-2-(N-propyl-N-2thienylethylamino) -5-hydroxy-tetraline (e.g. as N-0923) or (R)-5,6-dihydro -5-(methylamino)-4H-imidazo(4,5-ij)-quinolin-2(1H)-one and R-6 (PNU 95666) and the pharmacologically acceptable salts thereof.

13. The method according to claim 12, wherein the amount of bromocryptine corresponds to an oral dose of 1.25-20.0 mg; that of α-dihydroergocryptine corresponds to an oral dose of 5-60 mg; that of lisuride corresponds to an oral dose of 0.1-5 mg; that of pergolide corresponds to an oral dose of 0.05-1.0 mg; that of pramipexole (HCl) corresponds to an oral dose of 0.01-5.0 mg; and, that of ropinirol corresponds to an oral dose of 0.2-10.0 mg.

14. The method according to claim 12, wherein the dopamine agonist is selected from the group consisting of cabergoline, pergolide, piripedil, pramipexole, pramipexole hydrochloride, ropinirol, N-0923 and PNU 95666 and the pharmacologically acceptable salts thereof.

15. The method according to claim 12, wherein the dopamine agonist is pramipexole or pramipexole hydrochloride.

16. The method according to claim 1, wherein the second agent is an opioid.

17. The method according to claim 16, wherein the opioid is selected from the group consisting of buprenorphine, codeine, dextropropoxyphen, dihydrocodeine, fentanyl, hydromorphone, laevomethadone, morphine, oxycodon, pethidine, propoxyphen, sufentanyl, tilidine, tilidine/naloxone and tramadol and the pharmacologically acceptable salts thereof.

18. The method according to claim 17, wherein the quantity of codeine corresponds to an oral dose of 10 to 100 mg; that of dihydrocodeine corresponds to an oral dose of 10 to 100 mg; that of oxycodon corresponds to an oral dose of 4.5-20 mg; that of propoxyphen corresponds to an oral dose of 65-300 mg; that of tilidine/naloxone corresponds to an oral dose of 40-60/3-5 mg; that of tramadol corresponds to an oral dose of 10 to 500 mg; and that of morphine corresponds to an oral dose of 1 to 500 mg.

19. The method according to claim 1 wherein the second agent is a benzodiazepine or a pharmacologically acceptable salt thereof.

20. The method according to claim 18 wherein the benzodiazepine is selected from the group consisting of clonazepam, brotizolam, temazepam, nitrazepam and oxazepam or is a pharmacologically acceptable salt thereof.

21. The method according to claim 20, wherein the quantity of clonazepam corresponds to an oral dose of 0.01 to 10 mg; the quantity of brotizolam corresponds to an oral dose of 0.01 to 2 mg; that of temazepam corresponds to 15-30 mg; that of nitrazepam corresponds to 5-10 mg; and that of oxazepam corresponds to 10-20 mg.
